(19) United States
(12) Patent Application Publication
(10) Pub. No.: US 2019/0029449 A1
(43) Pub. Date: Jan. 31, 2019

(54) NOVELTY DEVICE AND METHOD FOR INFUSION, DRINKING AND INHALATION OF BOTANICALS

(71) Applicants: Angel Tonchev, Henderson, NV (US); Christo Tonchev, Henderson, NV (US)

(72) Inventors: Angel Tonchev, Henderson, NV (US); Christo Tonchev, Henderson, NV (US)

(21) Appl. No.: 16/051,648

(22) Filed: Aug. 1, 2018

Related U.S. Application Data
(60) Provisional application No. 62/538,709, filed on Jul. 30, 2017.

(51) Int. Cl. A47G 21/18; A61M 15/06

(52) U.S. Cl. CPC A47G 21/183; A24F 47/002; A61M 15/06

(57) ABSTRACT

We propose a novelty device and method for brewing, infusion, flavor extraction, drinking and inhalation of tea, herbs, coffee, and other botanicals. The device integrates an infuser, a straw, and an inhalator, into one compact, multi-functional device that uses a variable-pressure, multi-stage, multi-layer infusion and flavor extraction method. The invention creates a completely new experience allowing users to fully enjoy the flavors of botanical substances in both liquid and gas (vapor) forms. In addition, the device addresses some of the limitations in the existing packaging and usage of the abovementioned botanical substances as well as straw designs and liquid infusion methods.

### TECHNICAL FIELD OF THE INVENTION

The present invention relates to methods and devices for brewing, infusion and consumption of tea, herbs, coffee and other botanicals. The invention also relates to drinking straw designs and to a lesser extend to vaporizers and inhalators.

### BACKGROUND OF THE INVENTION

Tea and herbs have been used for thousands of years to infuse water and other liquids. Over the years many world cultures have developed deep-rooted traditions, procedures and tools for preparing and drinking of these plant liquids. As a result of this cultural heritage, recent innovations in the tea industry have been somehow limited. For example, the last major innovation in the field was the invention of the tea bag by Thomas Sullivan in 1908. It was a revolutionary idea providing ease and simplicity of brewing. Unfortunately, most tea innovation since then have been improvements in materials and shape of the tea bag. For example, William Hermanson invented heat-sealed paper tea bags in 1930, Lipton Tea Company patented the so-called “flo-thru” bag, which has four sides instead of two in 1952, Tetley launched the round tea bag in 1992, Brooke Bond invented the pyramid tea bag shape in 1997. In recent years, ‘tea socks’ have become popular amongst some full-leaf tea drinkers.

The coffee industry, on the other hand, has been a good example of innovation. For example, new roasting and brewing techniques, creative retail shops and packaging were developed. Some notable innovations in this area include: the French press invented in the 1850's; the Espresso machine created in 1822; Emerson's vacuum brewer invented in 1922; Nespresso brewing machine and capsules designed in 1996; “The Clover”, an automated French press, capable of making a hot beverage in less than 60 seconds introduced in 2006 by The Coffee Equipment Company; and the BKON brewing machine that infuses coffee, tea and other substances invented by Vastardis in 2013.

Drinking straws, first introduced by Sumerians around 3,000 B.C.E, have skyrocketed in popularity during the last century. There have been innovations in the materials, shape and functionality of the straw. In addition, numerous suggestions of integrating flavoring agents into straws have been proposed. For example, U.S. Pat. No. 6,109,538 by Villani et al.; U.S. Pat. No. 5,094,861 by D'Auguste et al.; U.S. Pat. No. 2,867,536 by Mead et al.; U.S. Pat. No. 3,620,770 by Harvey; U.S. Pat. No. 3,615,595 by Guttag; U.S. Pat. No. 4,921,713 by FolWer; U.S. Pat. No. Des. 332,198 by Goodman Jr.; US. Pat. No. US 2003/0203075 A1 by Sutherland et. al.; US. Pat. No. US 2003/0087005 A1 by Rothwell et. al.; and US. Pat. No. US 2003/0042324 A1 Sughrue et. al.

Presently, straws are mostly used for cold drinks as the majority of the designs do not address the discomfort and risk of mount burn when hot drinks are consumed. Nevertheless, there have been some suggestions to tackle these issues (e.g. U.S. Pat. No. 7,537,173 B1 by Kogan; U.S. Pat. No. 6,460,777 B2 by Float et. al.).

Another recent development is the increased use of vaporizers and e-cigarettes as a substitute to conventional tobacco smoking. Many devices with all shapes and forms have been introduced. Most of these devices are battery or gas heated and use glycerin-based flavoring agents. There are also dry herb vaporizers, which use conduction or convection methods to vaporize the herbs. While vaporizers and e-cigarettes are considerably better choice than the regular cigarettes they can still have adverse effects on our health.

All of the above-mentioned examples indicate that a need clearly exists for products and methods for brewing, infusion and consumption of botanical substances that provide innovative solutions, quality improvements and new customer experiences.

### SUMMARY OF THE INVENTION

The present invention can be described as a novelty device and method for brewing, infusion, flavor extraction, drinking and inhalation of tea, herbs, coffee and other botanicals. It integrates three devices, namely an infuser, a straw, and an inhalator, into one compact, multi-functional device that uses a variable-pressure, multi-stage, multi-layer infusion and flavor extraction method. The invention creates a completely new experience allowing users to fully enjoy the flavors of botanical substances in both liquid and gas (vapor) forms. The device features a nested design comprising:
a larger diameter elongated outer member having a central bore extending fully throughout with micro openings on the side wall allowing liquids and gases to pass through it;
a smaller diameter elongated inner rod, nested in said central bore of said larger diameter member, having a central bore extending fully throughout with micro openings on the side wall allowing liquids and gases to pass through it;
an infusion chamber, formed between said larger diameter member inner surface (bore side) and said rod exterior surface, accommodating tea, herbs, coffee and other botanicals;
caps closing said infusion chamber having a central bore allowing the flow of liquids and gases through said rod central bore;
a flow regulator nested in said central bore of said smaller diameter elongated rod regulating the flow of liquids and gases by opening and blocking said central bore of said smaller diameter elongated rod;
an infusion regulator, adjoining said larger diameter elongated member, regulating the infusion speed by opening and blocking said micro openings of said larger diameter elongated member; and
a temperature measuring device embedded in at least one said member, helping the user to optimize the brewing and infusion process as well as giving an indication on when the liquid is at desired temperature levels and comfortable to drink.

The proposed device can be used as a botanical brewer and infuser. In this case, the user immerses the device in a glass of liquid and the infusion starts. The device can also be conventionally used as a drinking straw wherein the user draws the liquid with it. Finally, the same device can be utilized as an inhalator. In this case the user pulls the device out of the liquid and inhales from it. The aroma extraction is immediate.

### BRIEF DESCRIPTION OF THE DRAWINGS

The invention with its main components is best understood when read in conjunction with the following drawings:

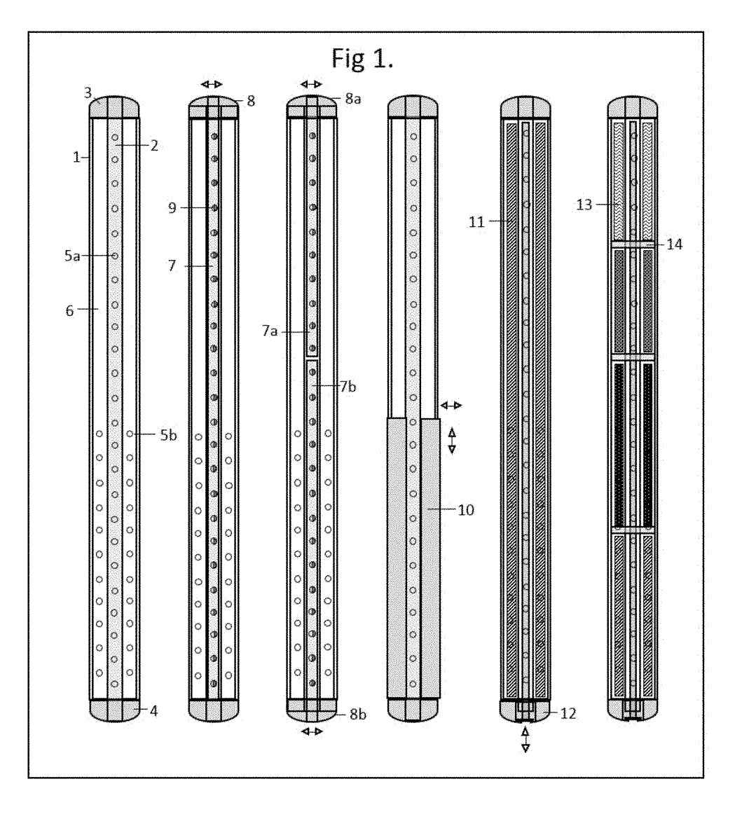

FIG. 1 is an illustration of the sectional view of the device and its members:
1. Larger diameter elongated outer member;
2. Smaller diameter elongated inner rod;
3. Top cap;
4. Bottom cap;
5a. Smaller diameter elongated inner rod micro openings;
5b. Larger diameter elongated outer member micro opening;
6. Infusion chamber;
7. Rod infusion regulator;
7a. Top rod infusion regulator;
7b. Bottom rod infusion regulator;
8. Infusion regulator cap regulating the infusion by twisting;
8a. Top infusion regulator cap regulating the infusion by twisting;
8b. Bottom infusion regulator cap regulating the infusion by twisting;
9. Rod micro opening valves;
10. Outer member infusion regulator opening and closing outer member micro openings by twisting and/or pulling movement;
11. Unstacked botanical substance;
12. Flow regulator;
13. Stacked botanical substances;
14. Separation O-rings.

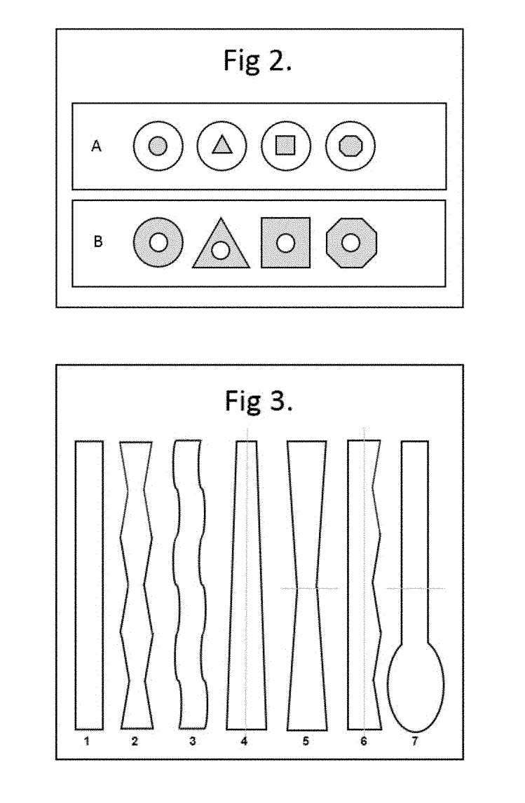

FIG. 2 is an illustration with cross-sectional examples for this device:
A. Smaller diameter elongated rod;
B. Larger diameter elongated member.

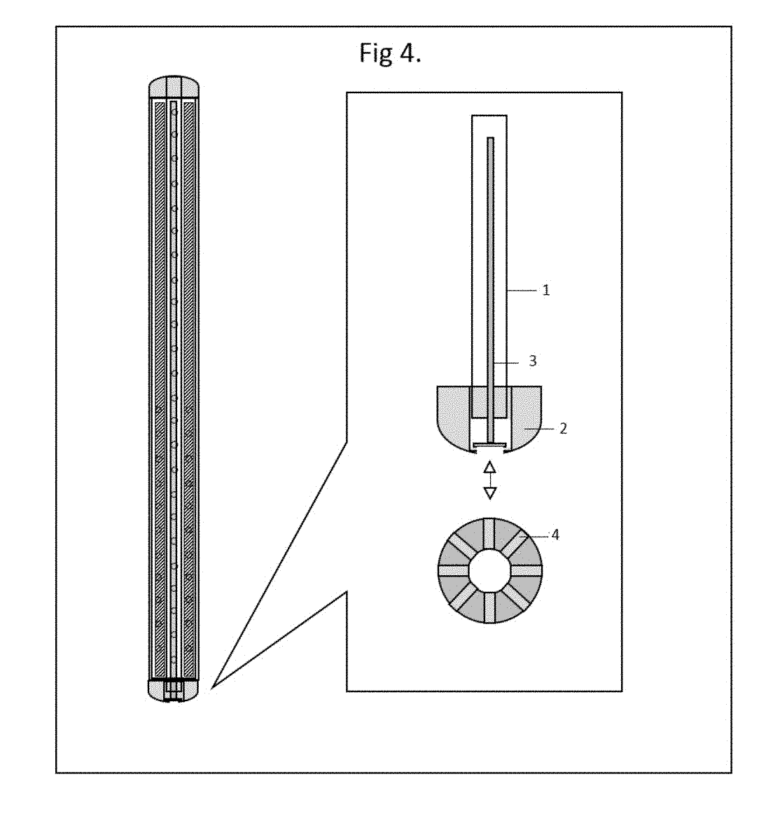

FIG. 3 is an illustration depicting examples of longitudinal cross-sectional shape for this device:
1. Rectangular shaped cross section;
2. Edgy cross section (example);
3. Curvy cross section (example);
4. Vertically symmetrical cross section (example);
5. Horizontally symmetrical cross section (example);
6. Vertically asymmetrical cross section (example);
7. Horizontally asymmetrical cross section (example).

FIG. 4 is an illustration depicting a flow regulator for this device and its members:
1. Smaller diameter elongated inner rod;
2. Flow regulator cap;
3. Flow regulator pin;
4. Flow regulator cap cuts, facilitating water and air flow.

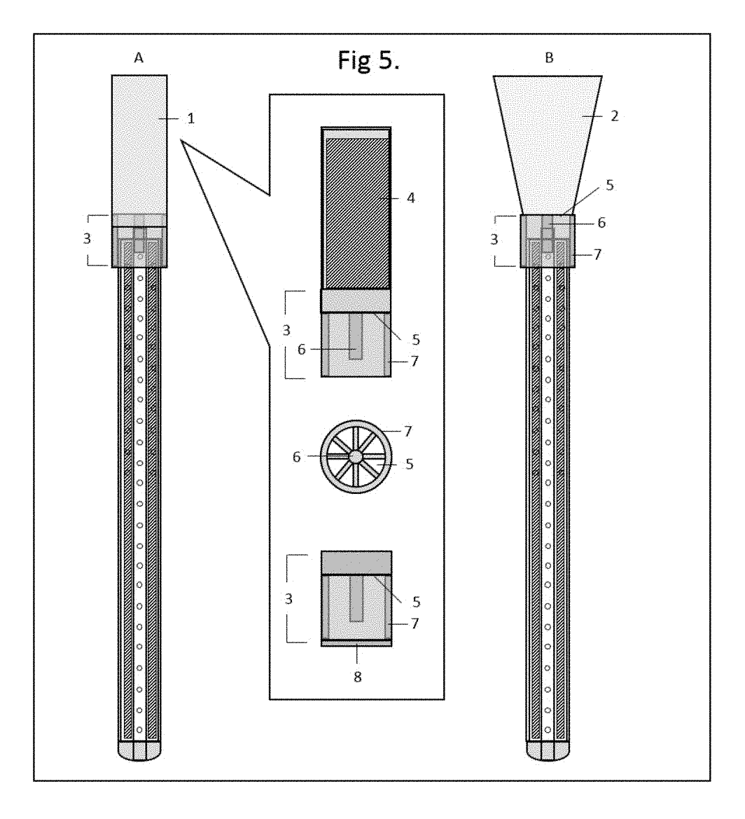

FIG. 5 is an illustration depicting a capsule and a funnel with their members to fill the device with botanicals:
A. Capsule;
B. Funnel;
1. Capsule body;
2. Funnel body;
3. Attachment end;
4. Botanical content;
5. Attachment end openings allowing botanicals to pour into the infusion chamber of the device;
6. Rod bore blocker, preventing botanicals to enter the central bore of the smaller diameter inner rod;
7. Attachment end external wall;
8. Attachment end stopper.

### DETAILED DESCRIPTION OF THE INVENTION

We propose a novelty device and method for brewing, infusion, flavor extraction, drinking and inhalation of tea, herbs, coffee, and other botanicals. The device integrates an infuser, a straw, and an inhalator, into one compact, multi-functional device that uses a variable-pressure, multi-stage, multi-layer infusion and flavor extraction method. The invention creates a completely new experience allowing users to fully enjoy the flavors of botanical substances in both liquid and gas (vapor) forms. In addition, the device addresses some of the limitations in the existing packaging and usage of the abovementioned botanical substances as well as straw designs and liquid infusion methods.

It has been a challenge for tea, herbs, coffee and other botanical substances that are in natural, grinded, shredded or dried form to be successfully integrated in a straw design. This is due to their specific properties and requirements, namely a volume expansion when soaked, a temperature requirement for infusion, a change of flavor when substances are compressed and a need for some sort of filtering. No previous art is known to successfully solve these issues in a straw design.

Our solution to these challenges is a compact device of nested members comprising:
a larger diameter elongated outer member having a central bore extending fully throughout with micro openings on the side wall allowing liquids and gases to pass through it;
a smaller diameter elongated inner rod, nested in said central bore of said larger diameter member, having a central bore extending fully throughout with micro openings on the side wall allowing liquids and gases to pass through it;
an infusion chamber, formed between said larger diameter member inner surface (bore side) and said rod exterior surface, accommodating tea, herbs, coffee and other botanicals;
caps closing said infusion chamber having a central bore allowing the flow of liquids and gases through said rod central bore;
a flow regulator nested in said central bore of said smaller diameter elongated rod regulating the flow of liquids and gases by opening and blocking said central bore of said smaller diameter elongated rod;
an infusion regulator, adjoining said larger diameter elongated member, regulating the infusion speed by opening and blocking said micro openings of said larger diameter elongated member; and
a temperature measuring device embedded in at least one said member, helping the user to optimize the brewing and infusion process as well as giving an indication on when the liquid is at desired temperature levels and comfortable to drink.

It should be noted that the flow and infusion regulators as well as the temperature meter add extra features to the device. However, the device can also work without them.

In addition, the following clarifications should be made:
the infusion chamber width can vary, however a balanced approach should be taken as increasing the width of the chamber will affect the liquid infusion and botanical compression rate which will ultimately have negative effect on flavor;
the infusion chamber design allows stacking of different layers of substances enabling a unique multi-layer brewing, infusion and aroma extraction;
O-rings can be used between stacked substances to separate and identify them;
the infusion chamber also acts as a heat insulator allowing the device to be comfortably held when drinking hot liquids;
the top (drawing) cap is preferably a convex or dome-shaped with bore diameter smaller that the diameter of the infusion chamber, reducing the flow of hot liquids and creating a broader surface area enabling better liquid temperature sensing;
the flow regulator is gravity and pressure operated allowing the flow of liquids and gases to pass through when the regulator is in upright position and the pressure in the rod bore equals the atmospheric pressure and blocking this flow when the regulator is flipped or negative pressure builds in the rod bore;
the flow regulator reduces the risks of mouth burns from hot liquids when they are drawn too fast and improves inhalation experience.

The proposed device can be used as a botanical brewer and infuser. In this case, the user first removes one of the infusion chamber caps and fills the chamber with botanical(s) (in stacks) with the help of a funnel. The user then closes the chamber with the cap and immerses the device into a reservoir of liquid so that a part of the device is above the liquid level. The infusion slowly begins in the section of the device that is below the liquid line. This is the initial first stage infusion, which is very similar to the one observed when a tea bag is placed in a glass of liquid.

The device can also be conventionally used as a drinking straw. In this case, the user starts drawing the liquid with it, creating a negative pressure inside the infusion chamber and rod bore pulling the slightly infused liquid and further infusing it with the botanical substance(s) above the liquid line. This is a second stage infusion characterized with a rapid extraction of volatile organic compounds (VOCs) which are the building blocks of the aroma. Because of the close proximity to our receptors and the encapsulation of these VOC gases the complexity and intensity of the aroma is enhanced. The liquid absorption with non-volatile compounds, which are associated with the sense of taste, is also enhanced because of the variable-pressure environment inside the infusion chamber. In particular, a cyclical reduction in pressure is observed when the liquid is drawn and subsequently left to balance with the atmospheric pressure.

The infusion can also be multi-layered. Instead of mixing all botanicals into one substance and blend their flavors we can stack them in the infusion chamber. This allows the substances to semi-independently brew and infuse, releasing complex blends of tastes and aromas. The type of substances, their volumes, layer positions and the speed of drawing all affect the final flavor.

Finally, the same device can be utilized as an inhalator. In this case the user pulls the device out of the water and starts inhaling from it. The aroma extraction is immediate.

What is claimed is:

1. A novelty device integrating an infuser, a straw and an inhalator into one multi-functional device for brewing, drinking and inhalation of tea, herbs, coffee and other botanicals, said device comprising:
a larger diameter elongated outer member having a central bore extending fully throughout with micro openings on the side wall that is made of one of the following materials: a glass material, a metal material, a plastic material, a silicone material, a ceramic material, a porcelain material, a wood material, a paper material, an organic material, and a multi-layer material combining said materials;
a smaller diameter elongated inner rod, nested in said central bore of said larger diameter member, having a central bore extending fully throughout with micro openings on the side wall that is made of one of the following materials: a glass material, a metal material, a plastic material, a silicone material, a ceramic material, a porcelain material, a wood material, a paper material, an organic material, and a multi-layer material combining said materials;
an infusion chamber, formed between said larger diameter member inner surface (bore side) and said rod exterior surface, accommodating tea, herbs, coffee and other botanicals; and
caps closing said infusion chamber, from top and bottom, having a central bore allowing the flow of liquids and gases through said rod central bore.

2. The invention of claim 1 wherein the horizontal cross-sectional shape of said larger diameter outer member and said smaller diameter inner rod is one cross-sectional shape selected from one of the following: a tubular shaped cross section, a square shaped cross section, a triangular shaped cross section, a hexagonal shaped cross section, and multi-angle cross section.

3. The invention of claim 1 wherein the longitudinal cross-sectional shape of said larger diameter outer member and said smaller diameter inner rod is one longitudinal cross-sectional shape selected from one of the following: a rectangular shaped cross section, an edgy cross section, a curvy cross section, a vertically symmetrical cross section, a horizontally symmetrical cross section, a vertically asymmetrical cross section, and a horizontally asymmetrical cross section.

4. The invention of claim 1 wherein said larger diameter outer member and said smaller diameter inner rod are merged and shaped (e.g. molded, extruded and 3D printed) into one member eliminating said infusion chamber cap(s).

5. The invention of claim 1 wherein said larger diameter elongated outer member is bended inwards at the end(s) closing said infusion chamber and eliminating said infusion chamber cap(s).

6. The invention of claim 1 wherein said smaller diameter elongated inner rod is bended outwards at the end(s) closing said infusion chamber and eliminating said infusion chamber cap(s).

7. The invention of claim 1 wherein said larger diameter outer member does not have micro openings on the side walls and is made of impermeable material blocking liquids and gases to pass through it.

8. The invention of claim 4 wherein said larger diameter outer member does not have micro openings on the side walls and is made of impermeable material blocking liquids and gases to pass through it.

9. The invention of claim 5 wherein said larger diameter outer member does not have micro openings on the side walls and is made of impermeable material blocking liquids and gases to pass through it.

10. The invention of claim 1 wherein said smaller diameter elongated inner rod is a plunger for cleaning said infusion chamber.

11. The invention of claim 1 wherein O-rings are placed between stacked botanical substances in said infusion chamber to separate and identify them.

12. The invention of claim 1 wherein a flow regulator is nested in said central bore of said smaller diameter elongated inner rod regulating the flow of liquids and gases by opening and blocking said central bore of said smaller diameter elongated inner rod.

13. The invention of claim 12 wherein said flow regulator is gravity and pressure operated allowing the flow of liquids and gases to pass through when said flow regulator is in upright position and the pressure in said central bore of said smaller diameter elongated rod equals the atmospheric pressure and blocking this flow when said flow regulator is flipped or negative pressure builds in said central bore of said smaller diameter elongated rod.

14. The invention of claim 1 wherein an infusion regulator is attached to said larger diameter elongated outer member regulating the speed of infusion by blocking and opening said sidewall micro openings of said larger diameter elongated outer member.

15. The invention of claim 1 wherein an infusion regulator is attached to said smaller diameter elongated inner rod regulating the speed of infusion by blocking and opening said sidewall micro openings of said smaller diameter elongated inner rod.

16. The invention of claim 1 wherein a temperature measuring device is embedded in at least one said member, helping the user to optimize the brewing and infusion process as well as giving an indication on when the liquid is at desired temperature levels and comfortable to drink.

17. The invention of claim 1 wherein a funnel is attached to said device to facilitate pouring of botanicals in said infusion chamber, said funnel comprising:
a funnel body accommodating botanicals that is with conical form and made of one of the following materials: a metal material, a plastic material, a silicone material, a ceramic material, a glass material, a porcelain material, a wood material, a paper material, an organic material, and a multi-layer material combining said materials;
an attachment end attaching to said device having a rod bore blocker, which blocks said central bore of said smaller diameter elongated rod, and openings, which allow botanicals from said funnel body to pass through and fill said infusion chamber;

18. A filling system for filling said infusion chamber of said device as described in claim 1 in the form of capsule, said capsule comprising:
a capsule body storing botanicals that is made of one of the following materials: a metal material, a plastic material, a silicone material, a ceramic material, a glass material, a porcelain material, a wood material, a paper material, an organic material, and a multi-layer material combining said materials;
an attachment end attaching to said device having a rod bore blocker, which blocks said central bore of said smaller diameter elongated rod, and openings, which allow botanicals from said capsule body to pass through and fill said infusion chamber;
an attachment end stopper closing said openings of said attachment end and sealing the content in said capsule.

19. The invention of claim 18 wherein said attachment end and said capsule body are merged and shaped (e.g. molded, extruded and 3D printed) into one member eliminating the need for connection.

20. A method for brewing, infusion and inhalation of botanicals, said method comprising the steps of:
providing a device having a larger diameter elongated outer member with a central bore extending fully throughout and micro openings on the side walls, a smaller diameter elongated inner rod nested in said central bore of said larger diameter outer member with a central bore extending fully throughout and micro openings on the side walls, an infusion chamber formed between said larger diameter member inner surface (bore side) and said rod exterior surface, and caps with a central bore closing said infusion chamber but allowing the flow of liquids and gases through said rod central bore;
filling said device infusion chamber with botanicals;
stacking layers of different botanicals into said device infusion chamber enabling multi-layer brewing;
immersing said device into a reservoir of liquid so that a part of said device is above said liquid level;
creating suction from said part of said device that is above said liquid level;
drawing continually until desired amount of said liquid from said reservoir passes through said device rod and infusion chamber and reaches user's mouth;
pulling said device completely out of said liquid from said reservoir; and
inhaling from said device for aroma inhalation.
